(19) United States
(12) Patent Application Publication
(10) Pub. No.: US 2005/0055018 A1
(43) Pub. Date: Mar. 10, 2005

(54) Method and device for sub-dermal tissue treatment

(76) Inventor: Michael Kreindel, Haifa (IL)

Correspondence Address: NATH & ASSOCIATES, 1030 15th STREET, NW, 6TH FLOOR, WASHINGTON, DC, 20005, US

(21) Appl. No.: 10/656,810

(22) Filed: Sep. 8, 2003

(51) Int. Cl. A61B 18/04

(52) U.S. Cl. 606/27

(57) ABSTRACT

System and method for non-invasive lysis of sub-dermal tissue by means of focused ultrasonic energy, the system comprising: a source of ultrasonic energy adapted to operate in continuous wave mode and to focus ultrasonic energy in a focal zone within the sub-dermal tissue, the ultrasonic energy being adapted to induce tissue cavitation in the focal zone; means for continuous displacement of the source over the skin surface; and means for determining a safe speed for the displacement, the safe speed allowing to avoid thermal tissue damage. The source of ultrasonic energy is accommodated in a hand-held applicator including a wheeled traction system powered by an electric drive.

### FIELD OF THE INVENTION

The invention relates to methods and devices for non-invasive ultrasonic destruction of living tissue in sub-dermal layer, in particular using CW focused ultrasonic energy.

### BACKGROUND OF THE INVENTION

During a long period, researchers have tried to use focused ultrasonic energy for non-invasive destruction of internal tissue. For example, U.S. Pat. No. 5,143,063 describes thermal fat destruction using focused ultrasonic energy. Parameters of the acoustic waves are adjusted to create thermal effect. The disadvantage of this method is the pain associated with thermal lysis of the living tissue.

Patent U.S. Pat. No. 5,624,382 describes thermal destruction of tumors. Cavitation is described there as a negative phenomenon, because bubbles created due to cavitation scatter ultrasonic energy and prevent its focusing.

U.S. Pat. No. 6,450,979 describes a device for lipolysis by continuous wave (CW) ultrasonic emission where safety is maintained by limiting ultrasonic energy intensity at a very low level, so as to avoid cavitation.

Thus, most devices invented for deep tissue necrosis such as removing of tumors or lipolysis have been designed for pure thermal effect without cavitation.

There are also devices trying to use cavitation induced by focused ultrasonic energy. U.S. Pat. No. 6,113,558 describes a device based on focused ultrasonic energy with parameters optimized for creating cavitation inside the tissue. The thermal damage is avoided by producing pulses shorter than 100 ms and duty cycle ratios over 5.

Patent application U.S. Ser. No. 2002/0082589 describes similar invention for lysis of adipose tissue using pulsed ultrasonic energy and cavitation, with duty cycle low enough to avoid thermal damage inside the skin. Detectors of cavitation are proposed for feed-back and control of a frame moving the transducer incrementally or continuously over the body and rupturing cells in a generally planar layer of adipose tissue.

### SUMMARY OF THE INVENTION

In accordance with one aspect of the present invention, there is provided a system for non-invasive lysis of sub-dermal tissue such as adipose (fat) tissue by means of focused ultrasonic energy comprising
(i) a source of ultrasonic energy adapted to operate in continuous wave mode and to focus ultrasonic energy in a focal zone within the sub-dermal layer tissue, the ultrasonic energy being adapted to induce tissue cavitation in the focal zone;
(ii) means for continuous displacement of the source over the skin surface; and
(iii) means for determining of a safe speed of the continuous displacement that allows obtaining the cavitation while avoiding thermal damage of the tissue.

The source of ultrasonic energy is preferably accommodated in a hand-held applicator, the displacement being provided by an operator.

The system has means for measurement of current speed and indication for adjustment of the current speed to the safe speed. The indication may be, for example, visual or audio indication.

The system may also comprise a powered traction system, for example with driving wheels, adapted to displace the applicator with the safe speed. The operator in this case only holds the applicator against the patient's body.

The system preferably comprises safety means adapted to limit the delivery of the ultrasonic energy if the safe speed is not maintained.

The source of ultrasonic energy is preferably a piezoelectric transducer. It may be shaped as a spherical transducer, cylindrical transducer, a phase array, Fresnel lens, etc. The transducer may be shaped for rolling over the skin surface.

According to another aspect of the present invention, there is provided a method for non-invasive lysis of sub-dermal tissue such as adipose (fat) tissue, comprising:
(i) Providing a source of continuous-wave ultrasonic energy with parameters adapted to induce tissue cavitation;
(ii) Determining a safe speed for continuous displacement of the source over the skin surface, such that the safe speed is slow enough to allow cavitation in the focus zone but fast enough to avoid thermal tissue damage;
(iii) Applying said source to the skin surface and focusing the ultrasonic energy in a focal zone at a predetermined depth under the skin; and
(iv) Displacing the source over the skin surface maintaining the safe speed.

The method further comprises measurement of current displacement speed of the source and indication for adjustment of the current speed towards the safe speed.

According to the method, the calculation of the safe speed depends on intensity of ultrasonic energy in the focus and the absorption coefficient of ultrasonic energy by the tissue.

For lysis of adipose tissue, the method preferably uses ultrasonic energy intensity at least 1000 mW/cm², frequency 0.5 to 1.5 MHz, focal zone dimension at the most 1 cm, and safety speed 0.2 to 2 cm/sec.

The present invention thus provides an apparatus and method for treatment of adipose or other tissue by continuous emission of focused ultrasound energy in the subcutaneous fat layer, for example at a depth of 0.3 to 3 cm, and adjusting the energy level to destroy fat cells by cavitation without thermal damage to the connective tissue and the blood vessels. This effect is achieved due to the continuous displacement of the source over the skin surface at a safe speed.

The apparatus advantageously calculates the safe speed of the source and provides indication for speed adjustment. The speed optimization allows usage of relatively high energy intensity over 1000 mW/cm² and effective destruction of the fat tissue without thermal damage or pain.

The accommodation of the source in a hand-held applicator provides portability and flexibility in the usage of the method. The powered traction system assists the operator to maintain the optimal safe speed and relieves his physical and psychical load which is very important for treatments like fat removal that require destruction of relatively large volume of adipose tissue.

### BRIEF DESCRIPTION OF THE DRAWINGS

In order to understand the invention and to see how it may be carried out in practice, preferred embodiments will now be described, by way of non-limiting examples only, with reference to the accompanying drawings, in which:

FIG. 1 shows a system for treatment of adipose tissue using focused CW ultrasonic energy in accordance with the invention;

FIG. 2 shows a cylindrical ultrasonic transducer with elongated focal zone;

FIG. 3 shows a hand-held applicator for adipose tissue treatment using focused CW ultrasonic energy in accordance with the invention; and

FIG. 4 shows an applicator with traction system and spherical ultrasonic transducer in accordance with the invention.

### DETAILED DESCRIPTION OF PREFERRED EMBODIMENTS

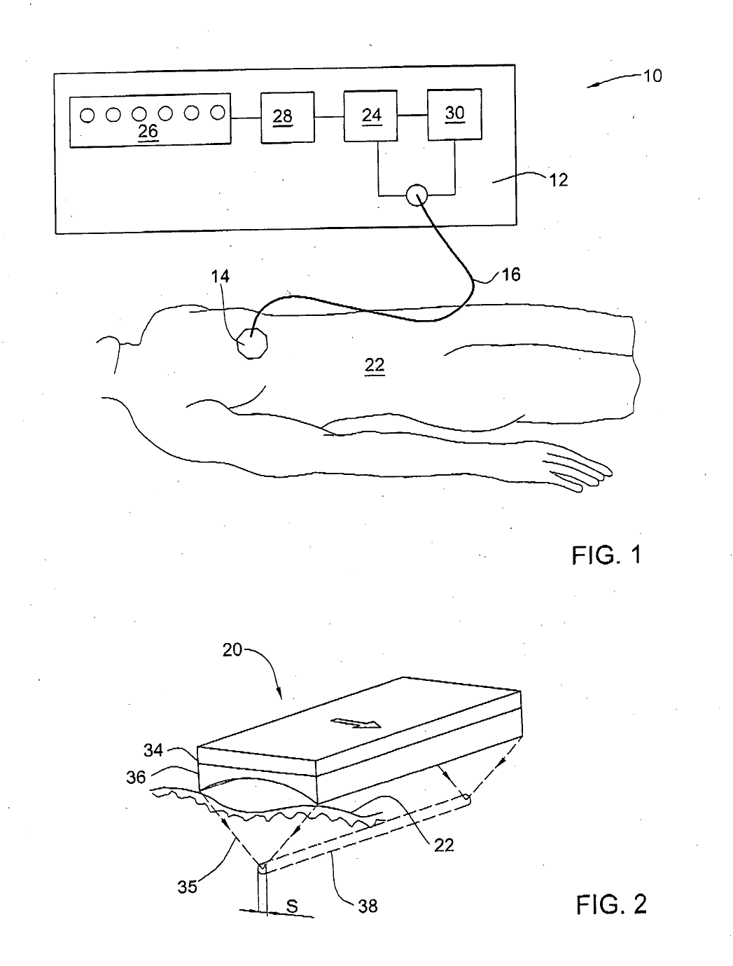

Referring now to FIG. 1, a system 10 for applying focused ultrasonic energy in accordance with the invention is shown. The system comprises a control unit 12, and an applicator 14 connected to the control unit by a cable 16. The applicator 14 contains a source of acoustic waves 20 (not seen in FIG. 1) and is adapted to be moved over the slain surface of an individual 22 in the treated region. The control unit 12 includes a power source 24 and control panel 26. The power source 24 generates high frequency electrical voltage and sends it to the applicator 14 via conductors in the cable 16. The control panel 26 is an input device that allows an operator to input selected values of parameters of the treatment, such as the ultrasonic energy intensity. The control unit 12 farther contains a processor 28 for monitoring and controlling various functions of the system, such as calculation of the safe speed, indicators for adjusting the current speed, etc. Included is also an electrical motor driver 30 to generate DC voltage for the motor of the traction system (see below).

FIG. 2 shows one possible design for a source of acoustic waves 20 comprising a piezoelectric ceramic transducer 34 and an acoustic cylindrical lens 36. The lens 36 is acoustically coupled to the transducer 34 and capable of focusing ultrasonic energy emitted by the transducer 34 (shown by rays 35) in a relatively narrow focal zone 38. This source can generate cylindrical acoustic wave with local zone under the skin surface 22 of the treated individual at the depth of 0.3 mm to 3 mm.

FIG. 3 shows in detail one embodiment of the applicator 14. The applicator comprises a carrying frame 40 with a handle 42, an ultrasound transducer 44 with polyurethane pads 46, and a drive unit 48, all mounted on the frame 40. The pads 46 are designed to provide acoustic coupling between the transducer 44 and the skin surface 22. The drive unit 48 has an electric motor with gear, while the transducer 44 is formed as a rolling wheel. The drive unit 48, the rolling wheel 44, and the electric driver 30 constitute the traction system of the applicator designed to provide continuous displacement. The applicator further has a sensor for measuring the displacement speed, which may be integrated with the motor or separate.

In operation, the operator turns on the control unit 12 and sets the energy intensity. The processor 28 calculates and indicates the safe speed of the applicator. The operator further takes the applicator by the handle 42, activates the traction system, and passes the rolling transducer 44 over the skin of the patient following the safe speed established by the traction system.

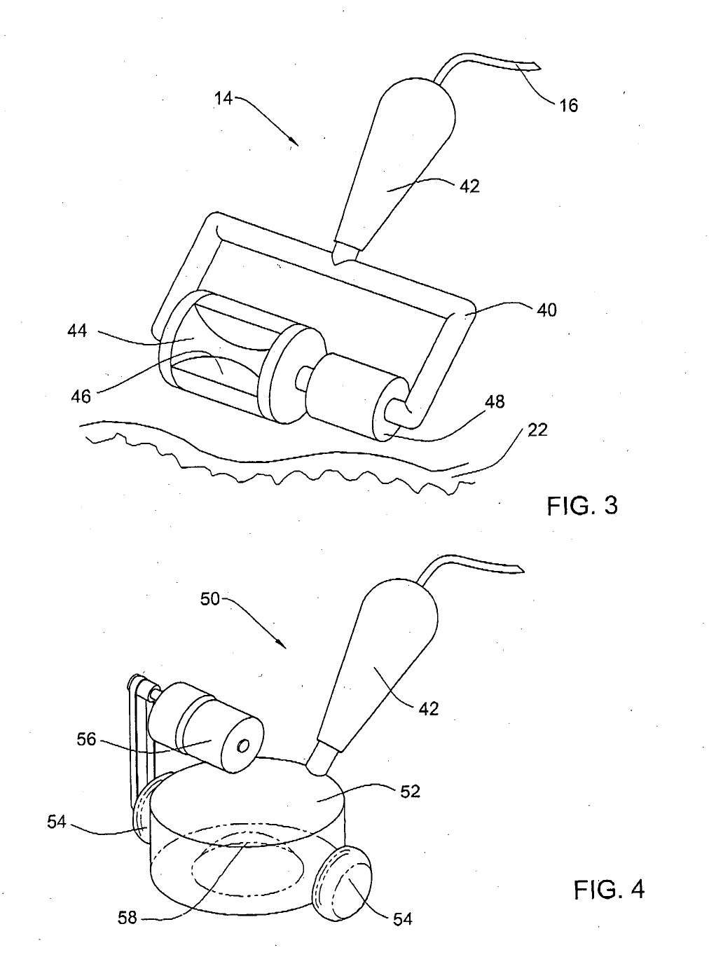

Another embodiment of the inventive system is presented in FIG. 4. An applicator 50 with cylinder shape accommodates a spherical transducer 52. The applicator has traction wheels 54 and DC motor 56. The spherical reflector 58 of the transducer focuses acoustic energy in sub-dermal level at the depth of 0.3 mm to 3 cm. The operator controls the direction of motion using the handle 42.

As a non-limiting example, the safe speed may be calculated in the following way. For given ultrasonic power density P[W/cm²] in the focal zone, and known absorption coefficient of ultrasound energy in the tissue A[cm⁻¹], the power absorption, D[W/cm³] will be:
D=PA

Noting specific heat of the tissue by c[J/(cm³*K)], and the tolerable (painless) temperature raise by ΔT[K], we obtain the maximal energy E that a unit volume of tissue may absorb as E=ΔT/c. The emitted energy in the focal zone for exposure time t is E=Dt. Thus, the maximal exposure time t_{MAX} will be

$${t}_{MAX} = \frac{Δ T}{cPA}$$

The minimal (safe) displacement speed V can be calculated as ratio of focus size S[m] in the displacement direction, and maximal exposure time:

$$V > \frac{S}{{t}_{MAX}} = \frac{ScPA}{Δ T}$$

The absorption coefficient of ultrasound energy in the tissue is known to be A=0.3 cm⁻¹. The specific heat of the tissue may be assumed similar to water c=4.2 [J/(cm³*K)], while the temperature raise should not exceed ΔT=10° K. Then, for a focus size S=0.8 cm and power density 10 W/cm², the safe speed will be about 1 cm/sec.

The optimal parameters for adipose tissue destruction were found to be:
Acoustic power generated by the transducer 0.3 to 3 kW
Focus depth 0.3 cm to 3 cm; Focus size should be not larger than 1 cm;
Transducer displacement speed 0.2 cm/sec to 2 cm/sec;
Energy density above 1000 mW/cm².
Preferable frequency range 0.1 to 1.5 MHz

Although a description of specific embodiments has been presented, it is contemplated that various changes could be made without deviating from the scope of the present invention. For example, the present invention could be used for lysis of other tissues than adipose tissue, suitably selecting parameters of treatment. The system may employ various indication means, both disposed in the control unit or in the applicator. The traction system may for example employ belts (caterpillars), etc.

1. A system for non-invasive lysis of sub-dermal tissue by means of focused ultrasonic energy, comprising:
(i) a source of ultrasonic energy adapted to operate in continuous wave mode and to focus ultrasonic energy in a focal zone within said sub-dermal tissue, said ultrasonic energy being adapted to induce tissue cavitation in said focal zone;
(ii) means for continuous displacement of said source over the skin surface; and
(iii) means for determining a safe speed for said continuous displacement, said safe speed allowing to obtain said cavitation while avoiding thermal damage of said tissue.

2. The system of claim 1, wherein said focal zone is at a depth of 0.3 to 3 cm under the skin surface.

3. The system of claim 1, wherein said tissue is adipose tissue.

4. The system of claim 1, further comprising means for measurement of current speed and indication for adjustment of said current speed to said safe speed.

5. The system of claim 4, wherein said indication is visual and/or audio indication.

6. The system of claim 1, wherein said source of ultrasonic energy is accommodated in a hand-held applicator.

7. The system of claim 6, wherein said means for continuous displacement includes a powered traction system.

8. The system of claim 7, wherein said traction system has driving wheels.

9. The system of claim 7, wherein said traction system is powered by an electric drive.

10. The system of claim 3, wherein said focused ultrasonic energy has intensity at least 1000 mW/cm².

11. The system of claim 3, wherein said source of ultrasonic energy is adapted to emit energy with frequency from 0.1 to 1.5 MHz.

12. The system of claim 3, wherein the dimension of said focal zone in the direction of displacement is not more than one centimeter.

13. The system of claim 3, wherein said means for displacement are adapted for safe speed between 0.2 and 2 cm/sec.

14. The system of claim 1, further comprising safety means adapted to limit delivery of said ultrasonic energy if said safe speed is not maintained.

15. The system of claim 1, wherein said source of ultrasonic energy is a piezoelectric transducer.

16. The system of claim 1, wherein said source of focused ultrasonic energy is a spherical transducer.

17. The system of claim 1, wherein said source of focused ultrasonic energy is a cylindrical transducer.

18. The system of claim 1, wherein said source of focused ultrasonic energy is a phase array.

19. The system of claim 1, wherein said source of ultrasonic energy has shape adapted for rolling over the skin surface.

20. A method for non-invasive lysis of sub-dermal tissue, said method comprising:
(i) Providing a source of continuous-wave ultrasonic energy with parameters adapted to induce tissue cavitation;
(ii) Determining a safe speed for continuous displacement of said source over the skin surface, such that said safe speed is slow enough to allow cavitation in the focus zone but fast enough to avoid thermal tissue damage;
(iii) Applying said source to the skin surface and focusing the ultrasonic energy in a focal zone at a predetermined depth under the skin; and
(iv) Displacing said source over the skin surface maintaining said safe speed.

21. The method of claim 20, wherein the calculation of said safe speed depends on the intensity of the ultrasonic energy in said focal zone and the ultrasonic energy absorption coefficient of said tissue.

22. The method of claim 20 further comprising measurement of current displacement speed of said source and indication for adjustment of said current speed towards said safe speed.

23. The method of claim 20, wherein said source of ultrasonic energy is accommodated in a hand-held applicator adapted for displacement by an operator.

24. The method of claim 23, wherein said hand-held applicator comprises a powered traction system for said displacement.

25. The method of claim 20, wherein said tissue is adipose tissue.

26. The method of claim 25, wherein said ultrasonic energy has intensity at least 1.000 mW/cm².

27. The method of claim 25, wherein said ultrasonic energy is emitted with frequency from 0.1. to 1.5 MHz.

28. The method of claim 25, wherein the dimension of said focal zone in the direction of displacement is not more than one centimeter.

29. The method of claim 25, wherein said safe speed is between 0.2 and 2 cm/sec.
